(19) United States
(12) Patent Application Publication
(10) Pub. No.: US 2025/0231107 A1
(43) Pub. Date: Jul. 17, 2025

(54) DIGITAL LOCALIZED SURFACE PLASMON RESONANCE SENSOR USING BUNDLE OF OPTICAL FIBERS AND FABRICATION METHOD

(71) Applicant: INDUSTRY-ACADEMIC COOPERATION FOUNDATION, DANKOOK UNIVERSITY, Yongin-si Gyeonggi-do (KR)

(72) Inventors: Seung Ki LEE, Seoul (KR); Jae Hyoung PARK, Yongin-si Gyeonggi-do (KR); Hyeong Min KIM, Paju-si Gyeonggi-do (KR); Seung Chul YANG, Anyang-si Gyeonggi-do (KR)

(21) Appl. No.: 18/853,507

(22) PCT Filed: Mar. 17, 2023
(86) PCT No.: PCT/KR2023/003561

§ 371 Date: Oct. 2, 2024

(30) Foreign Application Priority Data
May 26, 2022 (KR) 10-2022-0064867

(51) Int. Cl. G01N 21/552; G01N 33/543

(52) U.S. Cl. CPC G01N 21/554; G01N 33/54373; G01N 2201/08

(57) ABSTRACT

A digital localized surface plasmon resonance sensor using a bundle of optical fibers and a fabrication method. The digital localized surface plasmon resonance sensor can determine the sensor's output value by counting the number of optical fibers that output a signal greater than a certain size when the number of antigens bound to the antibody exceeds a predetermined number.

### TECHNICAL FIELD

Surface Plasmon Resonance (SPR) is a phenomenon that occurs due to the collective oscillation of free electrons when incident light interacts with a metal thin film, such as gold or silver, or with nanoparticles or nanostructures.

SPR enables real-time measurement of interactions between biomolecules without the need for specific labels. This makes it useful for biosensors that analyze protein chips and various biological reactions. SPR sensors may be applied to a wide range of measurements, including specific binding between proteins, by utilizing this phenomenon.

In particular, localized SPR (LSPR) sensors offer the advantage of high-sensitivity sensing at a low cost. However, traditional LSPR sensors have faced challenges in measuring substances at low concentrations.

### DISCLOSURE OF THE INVENTION

### Technical Goals

The present disclosure proposes a digital localized surface plasmon resonance (LSPR) sensor using an optical fiber bundle and a fabrication method.

The present disclosure proposes an LSPR sensor and a fabrication method. The LSPR sensor may measure low-concentration antigens by determining, as the output value of the sensor, the number of optical fibers outputting a signal of a predetermined magnitude or more when a preset number or more of antigens are bound to antibodies for each of a plurality of optical fibers included in an optical fiber bundle.

### Technical Solutions

According to an embodiment of the present disclosure, a digital localized surface plasmon resonance (LSPR) sensor includes an optical fiber bundle including a plurality of optical fibers, wherein a number of optical fibers is determined as an output value of the digital LSPR sensor, the optical fibers outputting a signal of a predetermined magnitude or more when a preset number of antigens are respectively bound to a plurality of antibodies present in the plurality of optical fibers.

An amino group may be disposed on a cross-section of a core layer of each of the plurality of optical fibers, a metal material may be connected to the amino group, and a plurality of antibodies capable of being bound to an antigen may be connected to a surface of the metal material.

A digital value of 1 may be determined for an optical fiber outputting a signal of a predetermined magnitude or more when a preset number or more of antigens are bound to the antibodies, and a digital value of 0 may be determined for an optical fiber not outputting a signal of a predetermined magnitude or more when less than a preset number of antigens are bound to the antibodies.

According to an embodiment of the present disclosure, a digital LSPR sensor includes an optical fiber bundle comprising a plurality of optical fibers, wherein an output value of a sensor may be determined based on a number of optical fibers outputting a signal of a predetermined magnitude or more when a preset number of antigens are bound to a plurality of first antibodies present in the plurality of optical fibers, and wherein the antigens may be bound to a second antibody connected to a metal material.

The plurality of first antibodies may be disposed on a cross-section of a core layer of each of the plurality of optical fibers, and the second antibody connected to the metal material may be bound to the antigens bound to the plurality of first antibodies.

A digital value of 1 may be determined for an optical fiber outputting a signal of a predetermined magnitude or more when a preset number or more of antigens are bound to the first antibodies, and a digital value of 0 may be determined for an optical fiber not outputting a signal of a predetermined magnitude or more when less than a preset number of antigens are bound to the first antibodies.

According to an embodiment of the present disclosure, a method of fabricating a digital LSPR sensor includes (i) configuring a plurality of optical fibers into a single optical fiber bundle, (ii) disposing an amino group on a cross-section of a core layer of each of the plurality of optical fibers, (iii) connecting a metal material to the amino group, and (iv) connecting, to a surface of the metal material, an antibody capable of being bound to an antigen.

According to an embodiment of the present disclosure, a method of fabricating a digital LSPR sensor includes (i) configuring a plurality of optical fibers into an optical fiber bundle, (ii) disposing an epoxy group on a cross-section of a core layer of each of the plurality of optical fibers, and (iii) connecting, to the epoxy group, a first antibody capable of being bound to an antigen, wherein the antigen may be bound to a second antibody connected to a metal material.

### Effects of the Invention

According to an embedment of the present disclosure low-concentration antigens may be measured by determining, as the output value of a sensor, the number of optical fibers outputting a signal of a predetermined magnitude or more when a preset number or more of antigens are bound to antibodies for each of a plurality of optical fibers included in an optical fiber bundle.

### BRIEF DESCRIPTION OF DRAWINGS

FIG. 1 is a diagram illustrating the principle of a localized surface plasmon resonance (LSPR) sensor, according to the first embodiment of the present disclosure.

FIG. 2 is an example of comparing a result of a digital LSPR sensor to a result of an analog LSPR sensor when a concentration is greater than or equal to a limit of detection, according to the first embodiment of the present disclosure.

FIG. 3 is an example of comparing a result of the digital LSPR sensor to a result of the analog LSPR sensor when a concentration is less than a limit of detection, according to the first embodiment of the present disclosure.

FIG. 4 is a diagram illustrating the principle of an LSPR sensor, according to the second embodiment of the present disclosure.

FIG. 5 is an example of comparing a result of a digital LSPR sensor to a result of an analog LSPR sensor when a concentration is greater than or equal to a limit of detection, according to the second embodiment of the present disclosure.

FIG. 6 is an example of comparing a result of a digital LSPR sensor to a result of an analog LSPR sensor when a concentration is less than a limit of detection, according to the second embodiment of the present disclosure.

### BEST MODE FOR CARRYING OUT THE INVENTION

Hereinafter, embodiments will be described in detail with reference to the accompanying drawings. The scope of the right, however, should not be construed as limited by the embodiments set forth herein. In the drawings, like reference numerals are used for like elements.

Various modifications may be made to the embodiments. Here, the embodiments are not to be construed as limited by the disclosure and should be construed as including all changes, equivalents, and replacements within the idea and the technical scope of the disclosure.

Terms, such as first, second, and the like, may be used herein to describe components. Each of these terminologies is not used to define an essence, order or sequence of a corresponding component but used merely to distinguish the corresponding component from other component(s). For example, a first component may be referred to as a second component, and similarly the second component may also be referred to as the first component.

The terminology used herein is for the purpose of describing particular embodiments only and is not intended to limit the embodiments. As used herein, the singular forms “a”, “an”, and “the” are intended to include the plural forms as well, unless the context clearly indicates otherwise. It will be further understood that the terms “comprises/comprising” and/or “includes/including” when used herein, specify the presence of stated features, integers, steps, operations, elements, components, or groups thereof, but do not preclude the presence or addition of one or more other features, integers, steps, operations, elements, components or groups thereof.

Unless otherwise defined, all terms used herein including technical or scientific terms have the same meaning as commonly understood by one of ordinary skill in the art to which this disclosure pertains. Terms, such as those defined in commonly used dictionaries, are to be interpreted as having a meaning that is consistent with their meaning in the context of the relevant art, and are not to be interpreted in an idealized or overly formal sense unless expressly so defined herein.

In addition, when describing the embodiments with reference to the accompanying drawings, like reference numerals refer to like components and a repeated description related thereto will be omitted. In the description of embodiments, detailed description of well-known related structures or functions will be omitted when it is deemed that such description will cause ambiguous interpretation of the present disclosure.

Hereinafter, embodiments will be described in detail with reference to the accompanying drawings.

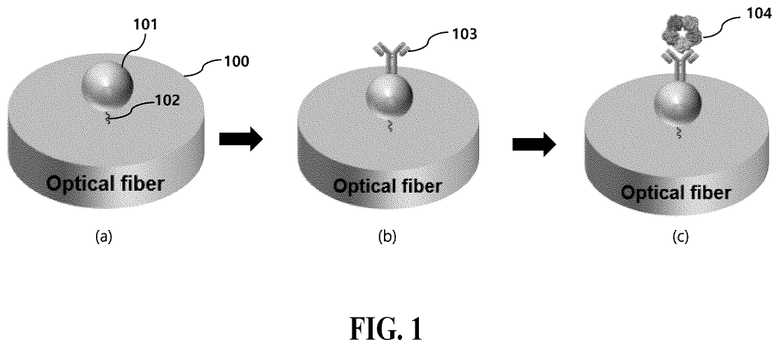

FIG. 1 is a diagram illustrating the principle of a localized surface plasmon resonance (LSPR) sensor, according to the first embodiment of the present disclosure.

LSPR may occur due to the interaction between the surface of a nanosized metal material and incident light injected into an optical fiber. When the incident light is directed onto the surface of the nanosized metal material that is smaller than the wavelength of the incident light, a resonance phenomenon may occur due to surface plasmon, which is a collective oscillation phenomenon of electrons caused by the interaction between the incident light and the electrons at the boundary between a metal surface and a dielectric at a predetermined wavelength. A refractive index may change due to an antigen bound to the top of an antibody fixed to the surface of the nanosized metal material, and as the refractive index changes, a shift in a resonance wavelength may occur. By utilizing the proportional relationship between the resonance wavelength shift and the concentration of the antigen bound to the top of the antibody fixed to the surface of the metal material, the concentration of the antigen bound to the antibody may be measured.

Referring to (a) of FIG. 1, an animo group 102 may be attached to the cross-section of an optical fiber 100. Then, a metal material 101, such as gold, may be attached to the amino group 102.

Referring to (b) of FIG. 1, an antibody 103 may be attached to the surface of the metal material 101.

Referring to (c) of FIG. 1, an antigen 104 may be bound to the antibody 103 attached to the surface of the metal material 101. In other words, the antibody 103 may be attached to the surface of the metal material 101 disposed on the cross-section of the optical fiber 100, and the antigen 104 to be measured may be bound to the antibody 103. The LSPR sensor according to an embodiment of the present disclosure may determine the concentration of the antigen 104 based on the resonance wavelength, which changes due to the antigen 104 bound to the antibody 103 disposed on the cross-section of the optical fiber 100 as the incident light progresses while undergoing total reflection within the optical fiber 100.

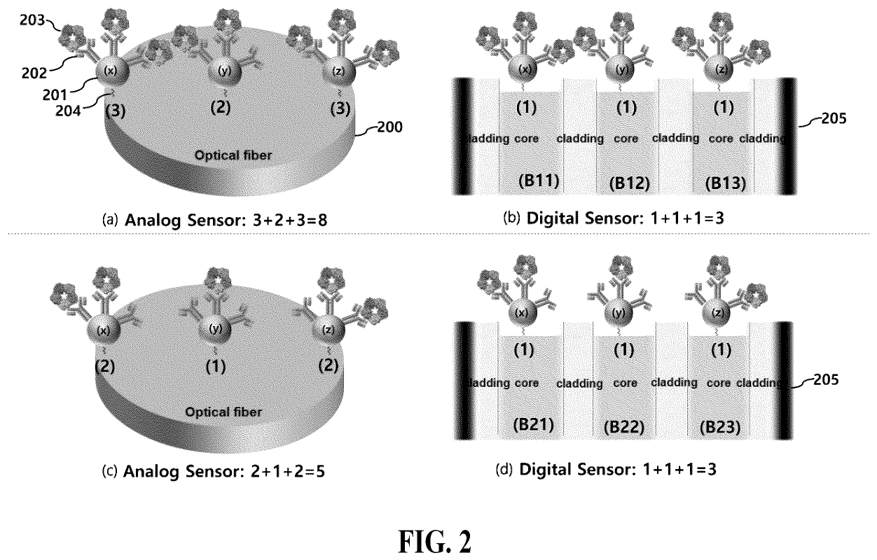

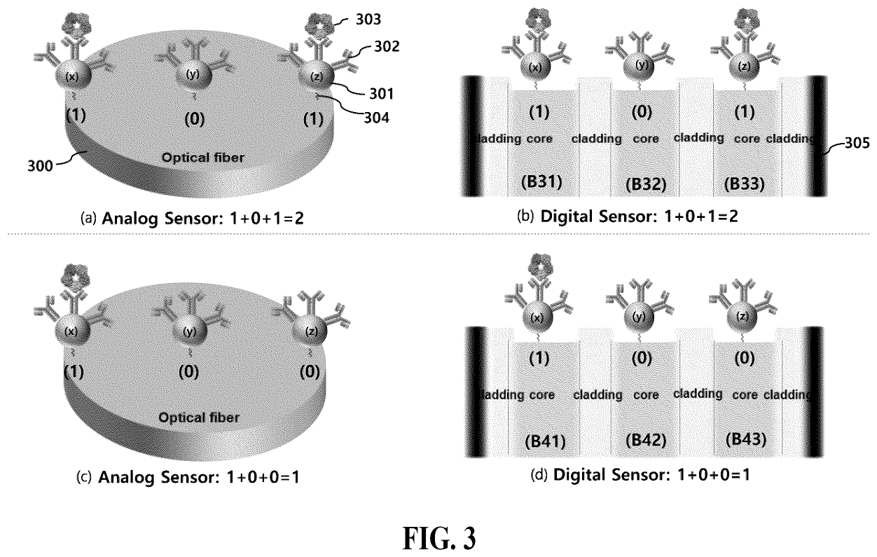

In particular, according to an embodiment of the present disclosure, as illustrated in FIGS. 2 and 3, the number of antigens 104 bound to the antibody 103 disposed on the cross-section of the core layer of the optical fiber 100 may be determined as the output value of an analog LSPR sensor. On the other hand, a digital LSPR sensor may configure a bundle of a plurality of optical fibers, and the number of optical fibers may be determined as the output value of the sensor, wherein the optical fibers output a signal of a predetermined magnitude or more when each of the plurality of optical fibers has the preset number or more of antigens 104 bound to the antibody 103. When the antigen 104 is not bound to the antibody 103 or when fewer than the preset number of antigens 104 are bound to the antibody 103, the optical fiber may not output a signal of a predetermined magnitude (or intensity) or more. Here, a signal of a predetermined magnitude or more may be generated when the preset number or more of antigens 104 are bound to the antibody 103.

FIG. 2 is an example of comparing a result of a digital LSPR sensor to a result of an analog LSPR sensor when a concentration is greater than or equal to a limit of detection, according to the first embodiment of the present disclosure.

(a) and (c) of FIG. 2 may represent analog LSPR sensors and (b) and (d) of FIG. 2 may represent digital LSPR sensors. An analog LSPR sensor may determine, as the output value of the sensor, a value proportional to the number of antigens 203 bound to an antibody 202 attached to the surface of a metal material 201. On the other hand, a digital LSPR sensor may determine, as the output value of the sensor, the number of optical fibers outputting a signal of a predetermined magnitude or more when a preset number or more of antigens 203 are bound to the antibody 202 attached to the surface of the metal material 201. When a preset number (threshold) or more of antigens 203 are bound to the antibody 202, a signal of a predetermined magnitude ore more is output, resulting in a digital value of 1. However, when the antigen 203 is not bound to the antibody 202 or when fewer than the preset number of antigens 203 are bound to the antibody 202, the optical fiber may output a signal less than a predetermined magnitude, resulting in a digital value of 0.

Referring to (a) of FIG. 2, an amino group 204 disposed on the cross-section of the core layer of an optical fiber 200 may be connected to metal materials 201 X, Y, and Z. Then, three antibodies 202 may be attached to the surface of each of the metal materials 201 X, Y, and Z. In (a) of FIG. 2, antigens 203 may be bound to all of the three antibodies 202 attached to the surface of the metal material 201 X. Then, in (a) of FIG. 2, antigens 203 may be bound to two of the three antibodies 202 attached to the surface of the metal material 201 Y. In addition, in (a) of FIG. 2, antigens 203 may be bound to all of the three antibodies 202 attached to the surface of the metal material 201 Z.

Referring to (a) of FIG. 2, the output value of the analog LSPR sensor may be determined to be a value proportional to 3+2+3=8 according to the number of antigens 203 based on the three antigens 203 bound to the antibodies 202 attached to the metal material 201 X, the two antigens 203 bound to the antibodies 202 attached to the metal material 201 Y, and the three antigens 203 bound to the antibodies 202 attached to the metal material 201 Z.

Referring to (c) of FIG. 2, the metal materials 201 X, Y, and Z may be connected to the amino group 204 disposed on the cross-section of the core layer of the optical fiber 200. Then, three antibodies 202 may be attached to the surface of each of the metal materials 201 X, Y, and Z. In (c) of FIG. 2, antigens 203 may be bound to the two antibodies 202 of the three antibodies 202 attached to the surface of the metal material 201 X. Then, in (c) of FIG. 2, an antigen 203 may be bound to one of the three antibodies 202 attached to the surface of the metal material 201 Y. In addition, in (c) of FIG. 2, antigens 203 may be bound to two antibodies 202 of the three antibodies 202 attached to the surface of the metal material 201 Z.

(c) of FIG. 2 may represent a scenario in which the concentration of the antigen 203 is lower compared to (a) of FIG. 2. Referring to (c) of FIG. 2, the output value of the analog LSPR sensor may be determined as a value proportional to 2+1+2=5 according to the number of antigens 203 based on the two antigens 203 bound to the antibodies 202 attached to the metal material 201 X, the one antigen 203 bound to the antibody 202 attached to the metal material 201 Y, and the two antigens 203 bound to the antibodies 202 attached to the metal material 201 Z.

Meanwhile, (b) of FIG. 2 illustrates a digital LSPR sensor that configures a plurality of optical fibers B11 to B13 into a single optical fiber bundle 205 and determines, as the output value of the sensor, the number of optical fibers outputting a signal of a predetermined magnitude or more when a preset number or more of antigens 203 are bound to the antibodies 202 in each of the plurality of optical fibers.

Referring to (b) of FIG. 2, the amino group 204 may be disposed on the cross- section of the core layer of each of the optical fiber B11, the optical fiber B12, and the optical fiber B13 included in the optical fiber bundle 205, and the amino group 204 may be connected to the metal materials 201 X, Y, and Z. Then, in (b) of FIG. 2, three antibodies 202 may be attached to each of the metal materials 201 X, Y, and Z. In (b) of FIG. 2, the metal material 201 X may be connected to the amino group 204 disposed on the cross-section of the core layer of the optical fiber B11, and antigens 203 may be bound to all of the three antibodies 202 attached to the surface of the metal material 201 X. Then, in (b) of FIG. 2, the metal material 201 Y may be connected to the amino group 204 disposed on the cross-section of the core layer of the optical fiber B12, and antigens 203 may be bound to two of the three antibodies 202 attached to the surface of the metal material 201 Y. In addition, in (b) of FIG. 2, the metal material 201 Z may be connected to the amino group 204 disposed on the cross-section of the core layer of the optical fiber B13, and antigens 203 may be bound to all of the three antibodies 202 attached to the surface of the metal material 201 Z.

Referring to (b) of FIG. 2, since antigens 203 are bound to the antibodies 202 attached to the metal material 201 X, antigens 203 are bound to the antibodies 202 attached to the metal material 201 Y, and antigens 203 are bound to the antibodies 202 attached to the metal material 201 Z, the output value of the digital LSPR sensor may be determined to be 1+1+1=3, which is the number of optical fibers outputting a signal of a predetermined magnitude or more when a preset number or more of antigens 203 are bound to the antibodies 202.

Meanwhile, (d) of FIG. 2 illustrates a digital LSPR sensor that configures a plurality of optical fibers B21 to B23 into a single optical fiber bundle 205 and determines, as the output value of the sensor, the number of optical fibers outputting a signal of a predetermined magnitude or more when a preset number or more of antigens 203 are bound to the antibodies 202 in each of the optical fibers.

Referring to (d) of FIG. 2, the metal materials 201 X, Y, and Z may be connected to the amino group 204 disposed on the surface of the optical fiber 200. Then, three antibodies 202 may be attached to the surface of each of the metal materials 201 X, Y, and Z. In (d) of FIG. 2, the metal material 201 X may be connected to the amino group 204 disposed on the cross-section of the core layer of the optical fiber B21, and antigens 203 may be bound to two antibodies 202 of the three antibodies 202 attached to the surface of the metal material 201 X. Then, in (d) of FIG. 2, the metal material 201 Y may be connected to the amino group 204 disposed on the cross-section of the core layer of an optical fiber B22, and an antigen 203 may be bound to one of the three antibodies 202 attached to the surface of the metal material 201 Y. In addition, in (d) of FIG. 2, the metal material 201 Z may be connected to the amino group 204 disposed on the cross-section of the core layer of the optical fiber B23, and antigens 203 may be bound to the two antibodies 202 of the three antibodies 202 attached to the surface of the metal material 201 Z.

Referring to (d) of FIG. 2, antigens 203 may be bound to the antibodies 202 attached to the metal material 201 X, an antigen 203 may be bound to the antibody 202 attached to the metal material 201 Y, and antigens 203 may be bound to the antibodies 202 attached to the metal material 201 Z. Therefore, the output value of the digital LSPR sensor may be determined to be 1+1+1=3, which is the number of optical fibers outputting a signal of a predetermined magnitude or more when a preset number or more of antigens 203 are bound to the antibodies 202.

In other words, according to the present disclosure, when a preset number or more of antigens 203 are bound to the plurality of antibodies 202, an output value of digital “1” may be generated, and when no antigens 203 are bound to the plurality of antibodies 202 or fewer than the preset number of antigens 203 are bound to the plurality of antibodies 202, an output value of digital “0” may be generated. If a limit of detection (limit of detector) concentration of the optical fiber 200 is a value proportional to 4, the analog LSPR sensor illustrated in FIG. 2 may determine the output value of the sensor to be a value proportional to 8, which exceeds the limit of detection concentration in (a) of FIG. 2, and a value proportional to 5 in (c) of FIG. 2.

FIG. 3 is an example of comparing a result of a digital LSPR sensor to a result of an analog LSPR sensor when a concentration is less than a limit of detection, according to the first embodiment of the present disclosure.

(a) and (c) of FIG. 3 may represent analog LSPR sensors and (b) and (d) may represent digital LSPR sensors. An analog LSPR sensor may use, as the output value of the sensor, a value proportional to the number of antigens 303 bound to antibodies 302 attached to the surface of a metal material 301. On the other hand, a digital LSPR sensor may determine, as the output value of the sensor, the number of optical fibers outputting a signal of a predetermined magnitude or more when a preset number or more of antigens 303 are bound to antibodies 302 attached to the surface of the metal material 301. Unlike FIG. 2, FIG. 3 illustrates a case in which the concentration of an antigen 303 is less than or equal to a limit of detection concentration. The analog LSPR sensors illustrated in (a) and (c) of FIG. 3 may not measure the concentration of an antigen 303 that is less than or equal to the limit of detection concentration.

Referring to (a) of FIG. 3, metal materials 301 X, Y, and Z may be connected to an amino group 304 disposed on the cross-section of the core layer of an optical fiber 300. Then, three antibodies 302 may be attached to the surface of each of the metal materials 301 X, Y, and Z. In (a) of FIG. 3, an antigen 303 may be bound to one antibody 302 of the three antibodies 302 attached to the surface of the metal material 301 X. Then, in (a) of FIG. 3, no antigen 303 may be bound to all of the three antibodies 302 attached to the surface of the metal material 301 Y. Furthermore, in (a) of FIG. 3, an antigen 303 may be bound to one antibody 302 of the three antibodies 302 attached to the surface of the metal material 301 Z.

Referring to (a) of FIG. 3, the output value of the analog LSPR sensor may be determined to be a value proportional to 1+0+1=2 according to the number of antigens 303 based on one antigen 303 bound to the antibody 302 attached to the metal material 301 X and one antigen 303 bound to the antibody 302 attached to the metal material 301 Z.

Referring to (c) of FIG. 3, the metal materials 301 X, Y, and Z may be connected to the amino group 304 disposed on the cross-section of the core layer of the optical fiber 300. Then, three antibodies 302 may be attached to the surface of each of the metal materials 301 X, Y, and Z. In (c) of FIG. 3, an antigen 303 may be bound to one antibody 302 of the three antibodies 302 attached to the surface of the metal material 301 X. Then, in (c) of FIG. 3, no antigens 303 may be bound to any of the three antibodies 302 attached to the surface of the metal material 301 Y. In addition, in (c) of FIG. 3, no antigen 303 may be bound to any of the three antibodies 302 attached to the surface of the metal material 301 Z.

Referring to (c) of FIG. 3, the output value of the analog LSPR sensor may be determined to be a value proportional to 1+0+0=1 according to the number of antigens 303 based on one antigen 303 bound to the antibody 302 attached to the metal material 301 X.

Meanwhile, (b) of FIG. 3 illustrates a digital LSPR sensor that configures a plurality of optical fibers B31 to B33 into a single optical fiber bundle 305 and determines, as an output value, the number of optical fibers outputting a signal of a predetermined magnitude by an antigen 303 bound to an antibody 302 in each of the optical fibers.

Referring to (b) of FIG. 3, the amino group 304 may be disposed on the cross-section of the core layer of each of the optical fiber B31, the optical fiber B32, and the optical fiber B33 included in the optical fiber bundle 305, and each of the metal materials 301 X, Y, and Z may be connected to the amino group 304. Then, three antibodies 302 may be attached to the surface of each of the metal materials 301 X, Y, and Z. In (b) of FIG. 3, the metal material 301 X may be connected to the amino group 304 disposed on the cross-section of the core layer of the optical fiber B31, and an antigen 303 may be bound to one antibody 302 of the three antibodies 302 attached to the surface of the metal material 301 X. Then, in (b) of FIG. 3, the metal material 301 Y may be connected to the amino group 304 disposed on the cross-section of the core layer of the optical fiber B32, and no antigen 303 may be bound to any of the three antibodies 302 attached to the surface of the metal material 301 Y. In addition, in (b) of FIG. 3, the metal material 301 Z may be connected to the amino group 304 disposed on the cross-section of the core layer of the optical fiber B33, and an antigen 303 may be bound to one antibody 302 of the three antibodies 302 attached to the surface of the metal material 301 Z.

Referring to (b) of FIG. 3, since one antigen 303 is bound to the antibody 302 attached to the metal material 301 X, no antigen 303 is bound to the antibodies 302 attached to the metal material 301 Y, and one antigen 303 is bound to the antibody 302 attached to the metal material 301 Z, the output value of the digital LSPR sensor may be determined to be 1+0+1=2, which is the number of optical fibers outputting a signal of a predetermined magnitude or more when a preset number or more of antigens 303 are bound to the antibodies 302. In (b) of FIG. 3, an optical fiber outputting a signal of a predetermined magnitude or more may be an optical fiber to which a preset number (e.g., 1) or more of antigens 303 are bound.

Meanwhile, (d) of FIG. 3 illustrates a digital LSPR sensor that configures a plurality of optical fibers B41 to B43 into a single optical fiber bundle 305 and determines, as the output value of the sensor, the number of optical fibers outputting a signal of a predetermined magnitude or more when a preset or more of antigens 303 are bound to antibodies 302 in each of the optical fibers.

Referring to (d) of FIG. 3, the metal materials 301 X, Y, and Z may be connected to the amino group 304 disposed on the surface of the optical fiber 300. Then, three antibodies 302 may be attached to the surface of each of the metal materials 301 X, Y, and Z. In (d) of FIG. 3, the metal material 301 X may be connected to the amino group 304 disposed on the cross-section of the core layer of the optical fiber B41, and an antigen 303 may be bound to one antibody 302 of the three antibodies 302 attached to the surface of the metal material 301 X. Then, in (d) of FIG. 3, the metal material 301 Y may be connected to the amino group 304 disposed on the cross-section of the core layer of the optical fiber B42, and no antigen 303 may be bound to any of the three antibodies 302 attached to the surface of the metal material 301 Y. In addition, in (d) of FIG. 3, the metal material 301 Z may be connected to the amino group 304 disposed on the cross- section of the core layer of the optical fiber B43, and no antigen 303 may be bound to any of the three antibodies 302 attached to the surface of the metal material 301 Z.

Referring to (d) of FIG. 3, since one antigen 303 is bound to the antibody 302 attached to the metal material 301 X and no antigens 303 are bound to the antibodies 302 of the metal material 301 Y and the metal material 301 Z, the output value of the digital LSPR sensor may be 1+0+0=1, which is the number of optical fibers outputting a signal of a predetermined magnitude or more when a preset number or more of antigens 303 are bound to the antibodies 302. In (d) of FIG. 3, an optical fiber outputting a signal of a predetermined magnitude or more may be an optical fiber to which a preset number (e.g., 1) or more of antigens 303 are bound.

In other words, according to the present disclosure, when a preset number or more of antigens 303 are bound to the plurality of antibodies 302, an output value of digital “1” may be generated, and when no antigens 303 are bound to any of the plurality of antibodies 302 or fewer than the preset number of antigens 303 are bound to the plurality of antibodies 302, an output value of digital “0” may be generated. If the limit of detection (limit of detector) concentration of the optical fiber 300 is a value proportional to 4, the analog LSPR sensors illustrated in FIG. 3 may not perform measurement because the concentration is less than the limit of detection concentration. However, the digital LSPR sensors illustrated in FIG. 3 may determine, as the output values of the sensors, the number of optical fibers outputting a signal of a predetermined magnitude or more when a preset number or more of antigens 303 are bound to the antibodies 302, regardless of the limit of detection concentration.

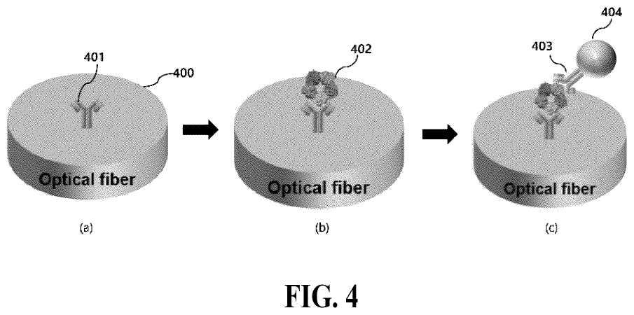

FIG. 4 is a diagram illustrating the principle of an LSPR sensor, according to the second embodiment of the present disclosure.

Referring to (a) of FIG. 4, a first antibody 401 may be attached to the cross-section of the core layer of an optical fiber 400. While the amino group 102 is attached to the cross-section of the core layer of the optical fiber 100 in (a) of FIG. 1, the first antibody 401 may be attached to the cross-section of the core layer of the optical fiber 400 in (a) of FIG. 4.

Referring to (b) of FIG. 4, an antigen 402 may be bound to the first antibody 401. Referring to (c) of FIG. 4, the antigen 402 bound to the first antibody 401 may be bound to a second antibody 403 connected to a metal material 404.

According to an embodiment of the present disclosure, incident light may progress within the core layer of the optical fiber 400 while undergoing total reflection and may be scattered by the metal material 404 connected to the second antibody 403 bound to the antigen 402 connected to the first antibody 401 on the cross-section of the core layer of the optical fiber 400. Then, the concentration of the antigen 402, which is the output value of an LSPR sensor, may be determined based on the intensity of the reflected light generated by the scattering of the incident light by the metal material 404.

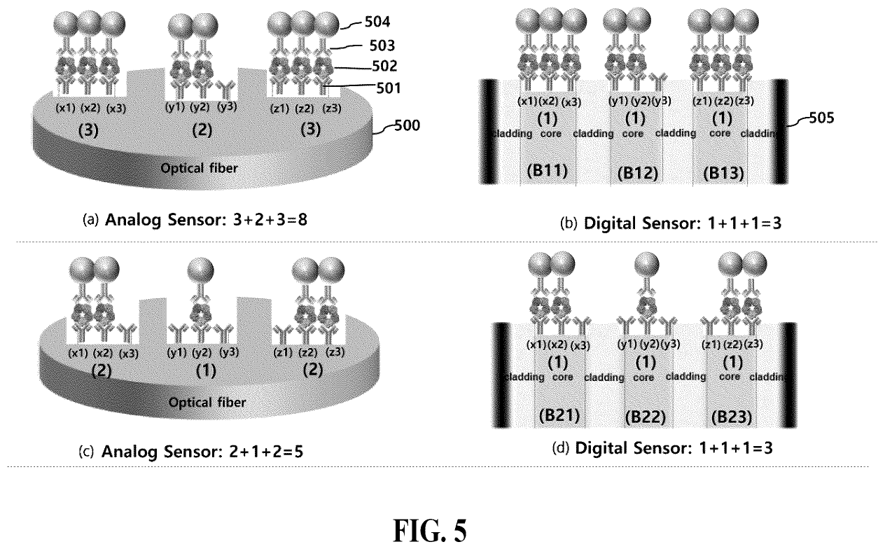

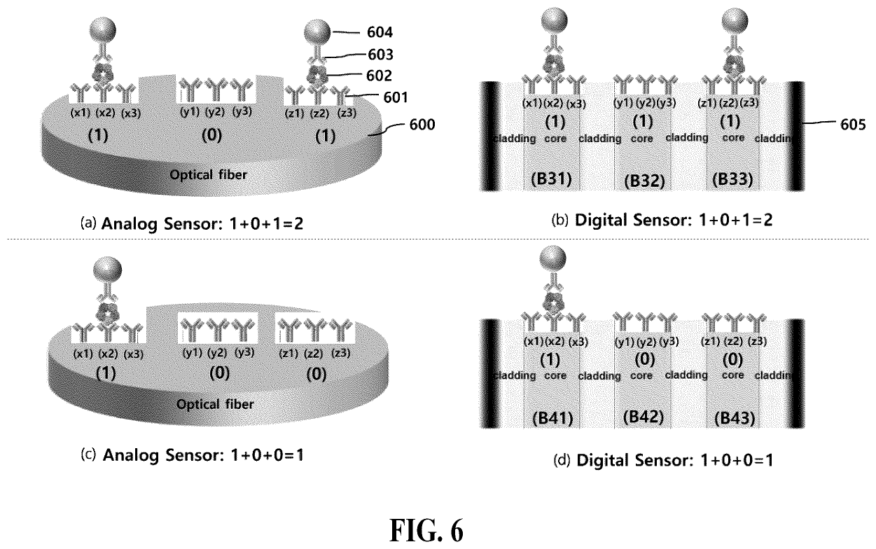

In particular, according to an embodiment of the present disclosure, FIGS. 5 and 6 illustrate an analog LSPR sensor that determines, as the output value of the sensor, a value proportional to the concentration of the antigen 402 bound to the first antibody 401 attached to the cross-section of the core layer of the optical fiber based on the intensity of the reflected light measured through a single optical fiber. In addition, illustrated is a digital LSPR sensor that determines, as the output value of the sensor, the number of optical fibers outputting a signal of a predetermined magnitude or more when a preset number or more of antigens 402 are bound to the first antibody 401 in a single optical fiber bundle including a plurality of optical fibers.

FIG. 5 is an example of comparing a result of a digital LSPR sensor to a result of an analog LSPR sensor when a concentration is greater than or equal to a limit of detection, according to the second embodiment of the present disclosure.

(a) and (c) of FIG. 5 may represent analog LSPR sensors and (b) and (d) of FIG. 5 may represent digital LSPR sensors. In an analog LSPR sensor, an antigen 502 may be bound to the surface of a first antibody 501 attached to the cross-section of the core layer of an optical fiber 500. Then, a second antibody 503 connected to a metal material 504 may be bound to the antigen 502. Therefore, an analog LSPR sensor may determine, as the output value of the sensor, a value proportional to the number of antigens 502 bound to first antibodies 501. On the other hand, a digital LSPR sensor may determine, as the output value of the sensor, the number of optical fibers outputting a signal of a predetermined magnitude or more when a preset number or more of antigens 502 are bound to first antibodies 501.

Referring to (a) of FIG. 5, first antibodies 501 X1 to X3, Y1 to Y3, and Z1 to Z3 may be disposed on the cross-section of the core layer of the optical fiber 500. Then, antigens 502 may be bound to the first antibodies 501 X1 to X3, Y1 to Y3, and Z1 to Z3. In (a) of FIG. 5, an antigen 502 may be bound to each of the first antibodies 501 X1 to X3. Then, a second antibody 503 connected to a metal material 504 may be bound to the antigen 502 bound to each of the first antibodies 501 X1 to X3. In addition, in (a) of FIG. 5, an antigen 502 may be bound to each of the first antibodies 501 Y1 and Y2. Then, a second antibody 503 connected to a metal material 504 may be bound to the antigen 502 bound to each of the first antibodies 501 Y1 and Y2. In (a) of FIG. 5, an antigen 502 may be bound to each of the first antibodies 501 Z1 to Z3. Then, a second antibody 503 connected to a metal material 504 may be bound to the antigen 502 bound to each of the first antibodies 501 Z1 to Z3.

Therefore, referring to (a) of FIG. 5, three antigens 502 may be bound to all of the first antibodies 501 X1 to X3 disposed on the cross-section of the core layer of the optical fiber 500, two antigens 502 may be bound to the first antibodies 501 Y1 and Y2, and three antigens 502 may be bound to all of the first antibodies 501 Z1 to Z3. Therefore, the output value of the analog LSPR sensor may be determined to be a value proportional to 3+2+3=8 according to the number of antigens 502 bound to the first 30 antibody 501.

Referring to (c) of FIG. 5, the first antibodies 501 X1 to X3, Y1 to Y3, and Z1 to Z3 may be disposed on the cross-section of the core layer of the optical fiber 500. Then, antigens 502 may be bound to the first antibodies 501 X1 to X3, Y1 to Y3, and ZI to Z3. In (c) of FIG. 5, an antigen 502 may be bound to each of the first antibodies 501 X1 and X2. Then, a second antibody 503 connected to a metal material 504 may be bound to the antigen 502 bound to each of the first antibodies 501 X1 and X2. In addition, in (c) of FIG. 5, an antigen 502 may be bound to the first antibody 501 Y2. Then, a second antibody 503 connected to a metal material 504 may be bound to the antigen 502 bound to the first antibody 501 Y2. In (c) of FIG. 5, an antigen 502 may be bound to each of the first antibodies 501 Z2 and Z3. Then, a second antibody 503 connected to a metal material 504 may be bound to the antigen 502 bound to each of the first antibodies 501 Z2 and Z3.

Therefore, referring to (c) of FIG. 5, two antigens 502 may be bound to the first antibodies 501 X1 and X2 disposed on the cross-section of the core layer of the optical fiber 500, one antigen 502 may be bound to the first antibody 501 Y2, and two antigens 502 may be bound to the first antibodies 501 Z2 and Z3. Therefore, the output value of the analog LSPR sensor may be determined to be a value proportional to 2+1+2=5 based on the number of antigens 502 bound to first antibodies 501.

Meanwhile, (b) of FIG. 5 illustrates a digital LSPR sensor that configures three optical fibers B11 to B13 into a single optical fiber bundle 505 and determines, as the output value, the number of optical fibers outputting a signal of a predetermined magnitude or more when a preset number or more of antigens are bound to the first antibodies 501 in each of the optical fibers.

Referring to (b) of FIG. 5, the first antibodies 501 X1 to X3, Y1 to Y3, and Z1 to Z3 may be respectively disposed on the cross-sections of the core layers of the optical fiber B11, the optical fiber B12, and the optical fiber B13 included in the optical fiber bundle 505. Then, antigens 502 may be bound to the first antibodies 501 X1 to X3, Y1 to Y3, and Z1 to Z3. Then, an antigen 502 may be bound to a second antibody 503 connected to a metal material 504.

In (b) of FIG. 5, an antigen 502 may be bound to each of the first antibodies 501 X1 to X3 disposed on the cross-section of the core layer of the optical fiber B11. Then, a second antibody 503 connected to a metal material 504 may be bound to the antigen 502 bound to each of the first antibodies 501 X1 to X3. In addition, in (b) of FIG. 5, an antigen 502 may be bound to each of the first antibodies 501 Y1 and Y2 disposed on the cross-section of the core layer of the optical fiber B12. Then, a second antibody 503 connected to a metal material 504 may be bound to the antigen 502 bound to each of the first antibodies 501 Y1 and Y2. In (b) of FIG. 5, an antigen 502 may be bound to each of the first antibodies 501 Z1 to Z3 disposed on the cross-section of the core layer of the optical fiber B13. Then, a second antibody 503 connected to a metal material 504 may be bound to the antigen 502 bound to each of the first antibodies 501 Z1 to Z3.

Referring to (b) of FIG. 5, three antigens 502 may be bound to the first antibodies 501 X1 to X3 disposed on the cross-section of the core layer of the optical fiber B11, two antigens 502 may be bound to the first antibodies 501 Y1 and Y2 disposed on the cross-section of the core layer of the optical fiber B12, and three antigens 502 may be bound to the first antibodies 501 Z1 to Z3 disposed on the cross-section of the core layer of the optical fiber B13. Therefore, the output value of the digital LSPR sensor illustrated in (b) of FIG. 5 may be 1+1+1=3, which is the number of optical fibers (the optical fiber B11, the optical fiber B12, and the optical fiber B13) outputting a signal of a predetermined magnitude or more when a preset number or more of antigens 502 are bound to the first antibodies 501 disposed on the cross-section of the core layer. For example, in (b) of FIG. 5, the preset number may be 1.

Referring to (d) of FIG. 5, the first antibodies 501 X1 to X3, Y1 to Y3, and Z1 to Z3 may be respectively disposed on the cross-sections of the core layers of the optical fiber B21, the optical fiber B22, and the optical fiber B23 included in the optical fiber bundle 505. Then, antigens 502 may be bound to the first antibodies 501 X1 to X3, Y1 to Y3, and Z1 to Z3. Then, an antigen 502 may be bound to a second antibody 503 connected to a metal material 504.

In (d) of FIG. 5, antigens 502 may be bound to the first antibodies 501 X1 and X2 disposed on the cross-section of the core layer of the optical fiber B21. Then, second antibodies 503 connected to metal materials 504 may be bound to the antigens 502 bound to the first antibodies 501 X1 and X2. In addition, in (d) of FIG. 5, an antigen 502 may be bound to the first antibody 501 Y2 disposed on the cross-section of the core layer of the optical fiber B22. Then, a second antibody 503 connected to a metal material 504 may be bound to the antigen 502 bound to the first antibody 501 Y2. In (d) of FIG. 5, an antigen 502 may be bound to each of the first antibodies 501 Z2 and Z3 disposed on the cross-section of the core layer of the optical fiber B23. Then, a second antibody 503 connected to a metal material 504 may be bound to the antigen 502 bound to each of the first antibodies 501 Z2 and Z3.

Referring to (d) of FIG. 5, two antigens 502 may be bound to the first antibodies 501 X1 and X2 disposed on the cross-section of the core layer of the optical fiber B21, one antigen 502 may be bound to the first antibody 501 Y2 disposed on the cross-section of the core layer of the optical fiber B22, and two antigens 502 may be bound to the first antibodies 501 Z2 and Z3 disposed on the cross-section of the core layer of the optical fiber B23. Therefore, the output value of the digital LSPR sensor illustrated in (d) of FIG. 5 may be determined to be 1+1+1=3, which is the number of optical fibers (the optical fiber B21, the optical fiber B22, and the optical fiber B23) outputting a signal of a predetermined magnitude or more when a preset number or more of antigens 502 are bound to the first antibodies 501 disposed on the cross-section of the core layer.

FIG. 6 is an example of comparing a result of a digital LSPR sensor to a result of an analog LSPR sensor when a concentration is less than a limit of detection, according to the second embodiment of the present disclosure.

(a) and (c) of FIG. 6 may represent analog LSPR sensors and (b) and (d) of FIG. 6 may represent digital LSPR sensors. Unlike FIG. 5, FIG. 6 illustrates a case in which an antigen 602 has a value less than or equal to a limit of detection concentration. The analog LSPR sensors illustrated in (a) and (c) of FIG. 6 may not measure the concentration of an antigen 602 less than or equal to the limit of detection concentration.

In an analog LSPR sensor, an antigen 602 may be bound to the surface of a first antibody 601 attached to the cross-section of the core layer of an optical fiber 600. Then, a second antibody 603 connected to a metal material 604 may be bound to the antigen 602. Therefore, the analog LSPR sensor may determine, as the output value of the sensor, a value proportional to the number of antigens 602 bound to first antibodies 601. However, a digital LSPR sensor may determine, as the output value of the sensor, the number of optical fibers outputting a signal of a predetermined magnitude or more when a preset number or more of antigens 602 are bound to first antibodies 601.

Referring to (a) of FIG. 6, first antibodies 601 X1 to X3, Y1 to Y3, and Z1 to Z3 may be disposed on the cross-section of the core layer of the optical fiber 600. Then, antigens 602 may be bound to the first antibodies 601 X1 to X3, Y1 to Y3, and Z1 to Z3. In (a) of FIG. 6, an antigen 602 may be bound to the first antibody 601 X2. Then, a second antibody 603 connected to a metal material 604 may be bound to the antigen 602 bound to the first antibody 601 X2. In addition, in (a) of FIG. 6, no antigens 602 may be bound to any of the first antibodies 601 Y1 to Y3. In (a) of FIG. 6, an antigen 602 may be bound to the first antibody 601 Z2. Then, a second antibody 603 connected to a metal material 604 may be bound to the antigen 602 bound to the first antibody 601 Z2.

Therefore, referring to (a) of FIG. 6, one antigen 602 may be bound to the first antibody 601 X2 disposed on the cross-section of the core layer of the optical fiber 600, no antigens 602 may be bound to any of the first antibodies 601 Y1 to Y3, and one antigen 602 may be bound to the first antibody 601 Z2. Therefore, the output value of the analog LSPR sensor may be a value proportional to 1+0+1=2 according to the number of antigens 602 bound to the first antibodies 601.

Referring to (c) of FIG. 6, the first antibodies 601 X1 to X3, Y1 to Y3, and Z1 to Z3 may be disposed on the cross-section of the core layer of the optical fiber 600. Then, an antigen 602 may be bound to the first antibodies 601 X1 to X3, Y1 to Y3, and Z1 to Z3. In (c) of FIG. 6, the antigen 602 may be bound to the first antibody 601 X2. Then, a second antibody 603 connected to a metal material 604 may be bound to the antigen 602 bound to the first antibody 601 X2. In addition, in (c) of FIG. 6, no antigens 602 may be bound to any of the first antibodies 601 Y1 to Y3. Furthermore, no antigens 602 may be bound to any of the first antibodies 601 Z1 to Z3.

Therefore, referring to (c) of FIG. 6, one antigen 602 may be bound to the first antibody 601 disposed on the cross-section of the core layer of the optical fiber 600, no antigens 602 may be bound to any of the first antibodies 601 Y1 to Y3, and no antigens 602 may be bound to any of the first antibodies 601 Z1 to Z3. Therefore, the output value of the analog LSPR sensor may be a value proportional to 1+0+0=2 according to the number of antigens 602 bound to the first antibodies 601.

Meanwhile, (b) of FIG. 6 illustrates a digital LSPR sensor that configures three optical fibers B31 to B33 into a single optical fiber bundle 605 and determines, as the output value of the sensor, the number of optical fibers outputting a signal of a predetermined magnitude or more when a preset number or more of antigens 602 are bound to the first antibodies 601.

Referring to (b) of FIG. 6, the first antibodies 601 X1 to X3, Y1 to Y2, and Z1 to Z3 may be respectively disposed on the cross-sections of the core layers of the optical fiber B31, the optical fiber B32, and the optical fiber B33 included in the optical fiber bundle 605. Then, antigens 602 may be bound to the first antibodies 601 X1 to X3, Y1 to Y3, and Z1 to Z3. Then, an antigen 602 may be bound to a second antibody 603 connected to a metal material 604.

In (b) of FIG. 6, an antigen 602 may be bound to the first antibody 601 X2 disposed on the cross-section of the core layer of the optical fiber B31. Then, a second antibody 603 connected to a metal material 604 may be bound to the antigen 602 bound to the first antibody 601 X2. In addition, in (b) of FIG. 6, no antigens 602 may be bound to any of the first antibodies 601 Y to Y3 disposed on the cross-section of the core layer of the optical fiber B32. In (b) of FIG. 6, an antigen 602 may be bound to the first antibody 601 Z2 disposed on the cross-section of the core layer of the optical fiber B33. A second antibody 603 connected to a metal material 604 may be bound to the antigen 602 bound to the first antibody 601 Z2.

Referring to (b) of FIG. 6, one antigen 602 may be bound to the first antibody 601 X2 disposed on the cross-section of the core layer of the optical fiber B31, no antigens 602 may be bound to any of the first antibodies 601 Y1 to Y3 disposed on the cross-section of the core layer of the optical fiber B32, and one antigen 602 may be bound to the first antibody 601 Z2 disposed on the cross-section of the core layer of the optical fiber B33. Therefore, the output value of the digital LSPR sensor illustrated in (b) of FIG. 6 may be 1+0+1=2, which is the number of optical fibers (the optical fiber B31 and the optical fiber B33) outputting a signal of a predetermined magnitude or more when a preset number (1 in the case of (b) of FIG. 6) or more of antigens 602 are bound to the first antibodies 601 disposed on the cross-section of the core layer.

Referring to (d) of FIG. 6, the first antibodies 601 X1 to X3, Y1 to Y3, and Z1 to Z3 may be respectively disposed on the cross-sections of the core layers of the optical fiber B41, the optical fiber B42, and the optical fiber B43 included in the optical fiber bundle 605. Then, an antigen 602 may be bound to the first antibodies 601 X1 to X3, Y1 to Y3, and Z1 to Z3. Then, the antigen 602 may be bound to a second antibody 603 connected to a metal material 604.

In (d) of FIG. 6, the antigen 602 may be bound to the first antibody 601 X2 disposed on the cross-section of the core layer of the optical fiber B41. Then, the second antibody 603 connected to the metal material 604 may be bound to the antigen 602 bound to the first antibody 601 X2. In addition, in (d) of FIG. 6, no antigens 602 may be bound to any of the first antibodies 601 Y1 to Y3 disposed on the cross-section of the core layer of the optical fiber B42. In (d) of FIG. 6, no antigens 602 may be bound to the first antibodies 601 Z1 to Z3 disposed on the cross-section of the core layer of the optical fiber B43.

Referring to (d) of FIG. 6, one antigen 602 may be bound to the first antibody 601 X2 disposed on the cross-section of the core layer of the optical fiber B41, no antigens 602 may be bound to any of the first antibodies 601 Y1 to Y3 disposed on the cross-section of the core layer of the optical fiber B42, and no antigens 602 may be bound to any of the first antibodies 601 Z1 to Z3 disposed on the cross-section of the core layer of the optical fiber B43. Therefore, the output value of the digital LSPR sensor illustrated in (d) of FIG. 6 may be 1+0+0=1, which is the number of optical fibers (the optical fiber B41) outputting a signal of a predetermined magnitude or more when a preset number (1 in the case of (d) of FIG. 6) or more of antigens 602 are bound to the first antibody 601 disposed on the cross-section of the core layer.

While the present specification contains many specific implementation details, these should not be construed as limitations on the scope of any disclosure or of what may be claimed, but rather as descriptions of features that may be specific to particular embodiments of particular disclosures. Specific features described in the present specification in the context of individual embodiments may also be combined and implemented in a single embodiment. On the contrary, various features described in the context of a single embodiment may be implemented in a plurality of embodiments individually or in any appropriate sub-combination. Moreover, although features may be described above as acting in specific combinations and even initially claimed as such, one or more features from a claimed combination can in some cases be excised from the combination, and the claimed combination may be changed to a sub-combination or a modification of a sub-combination.

Likewise, although operations are depicted in a predetermined order in the drawings, it should not be construed that the operations need to be performed sequentially or in the predetermined order, which is illustrated to obtain a desirable result, or that all of the shown operations need to be performed. In some cases, multi-tasking and parallel processing may be advantageous. In addition, it should not be construed that the division of various device components of the aforementioned example embodiments is required in all types of embodiments, and it should be understood that the described program components and devices are generally integrated as a single software product or packaged into a multiple-software product.

The embodiments disclosed in the present specification and the drawings are intended merely to present specific examples in order to aid in understanding of the present disclosure, but are not intended to limit the scope of the present disclosure. It will be apparent to those skilled in the art that various modifications based on the technical spirit of the present disclosure, as well as the disclosed embodiments, may be made.

1. A digital localized surface plasmon resonance (LSPR) sensor comprising:
an optical fiber bundle comprising a plurality of optical fibers,
wherein a number of optical fibers is determined as an output value of the digital LSPR sensor, the optical fibers outputting a signal of a predetermined magnitude or more when a preset number of antigens are respectively bound to a plurality of antibodies present in the plurality of optical fibers.

2. The digital LSPR sensor of claim 1, wherein
an amino group is disposed on a cross-section of a core layer of each of the plurality of optical fibers,
a metal material is connected to the amino group, and
a plurality of antibodies capable of being bound to an antigen is connected to a surface of the metal material.

3. The digital LSPR sensor of claim 1, wherein
a digital value of 1 is determined for an optical fiber outputting a signal of a predetermined magnitude or more when a preset number or more of antigens are bound to the antibodies, and
a digital value of 0 is determined for an optical fiber not outputting a signal of a predetermined magnitude or more when less than a preset number of antigens are bound to the antibodies.

4. A digital localized surface plasmon resonance (LSPR) sensor comprising:
an optical fiber bundle comprising a plurality of optical fibers,
wherein an output value of a sensor is determined based on a number of optical fibers outputting a signal of a predetermined magnitude or more when a preset number of antigens are bound to a plurality of first antibodies present in the plurality of optical fibers, and
wherein the antigens are bound to a second antibody connected to a metal material.

5. The digital LSPR sensor of claim 4, wherein
the plurality of first antibodies is disposed on a cross-section of a core layer of each of the plurality of optical fibers, and
the second antibody connected to the metal material is capable of being bound to the antigens bound to the plurality of first antibodies.

6. The digital LSPR sensor of claim 4, wherein
a digital value of 1 is determined for an optical fiber outputting a signal of a predetermined magnitude or more when a preset number or more of antigens are bound to the first antibodies, and
a digital value of 0 is determined for an optical fiber not outputting a signal of a predetermined magnitude or more when less than a preset number of antigens are bound to the first antibodies.

7. A method of fabricating a digital localized surface plasmon resonance (LSPR) sensor, the method comprising:
(i) configuring a plurality of optical fibers into a single optical fiber bundle;
(ii) disposing an amino group on a cross-section of a core layer of each of the plurality of optical fibers;
(iii) connecting a metal material to the amino group; and
(iv) connecting, to a surface of the metal material, an antibody capable of being bound to an antigen.

8. (canceled)
